(19) United States
(12) Patent Application Publication
(10) Pub. No.: US 2017/0209285 A1
(43) Pub. Date: Jul. 27, 2017

(54) INTERVERTEBRAL FUSION CAGE COMBINED WITH FIXING BLADE

(71) Applicant: IMI Co., Ltd., Ansan-si (KR)

(72) Inventors: Seong Heon Kim, Ansan-si (KR); Dong Wook Kim, Hwaseong-si (KR); Eun Mi Jung, Seoul (KR); Sam Sun Park, Seoul (KR); Hong Won Yoon, Yongin-si Gyeonggi-do (KR)

(21) Appl. No.: 15/004,182

(22) Filed: Jan. 22, 2016

(51) Int. Cl. A61F 2/44

(52) U.S. Cl. CPC A61F 2/4465; A61F 2002/4475; A61F 2220/0016; A61F 2002/449

(57) ABSTRACT

Provided is an intervertebral fusion cage combined with a fixing blade. The intervertebral fusion cage combined with the fixing blade, which is inserted to vertebral bodies adjacent to each other, includes a fusion cage body having a bone fusion hole passing from a top surface to a bottom surface thereof, the fusion cage body being inserted between the vertebral bodies adjacent to each other, an upper vertebral body coupling unit coupled to a first accommodation part defined inward from one side of a front portion of the fusion cage body, the upper vertebral body coupling unit having a lower portion that is coupled to the first accommodation part and an upper portion that is exposed to an upper side of the fusion cage body and provided with a first fixing blade inserted into an upper vertebral body, and a lower vertebral body coupling unit coupled to a second accommodation part defined inward from the other side of the front portion of the fusion cage body, the lower vertebral body coupling unit having an upper portion that is coupled to the second accommodation part and a lower portion that is exposed to a lower side of the fusion cage body and provided with a second fixing blade inserted into a lower vertebral body.

### STATEMENT REGARDING PRIOR DISCLOSURES

The present application claims the grace period exception under AIA 35 USC 102(b)(1)(A) to Korean Patent Registration No. 10-1498013 (published on Mar. 3, 2015), which is hereby incorporated by reference in its entirety.

### BACKGROUND

The present disclosure relates to an intervertebral fusion cage combined with a fixing blade, and more particularly, to an intervertebral fusion cage that fixes the fusion cage inserted between vertebral bodies by using a fixing blade provided thereon.

In general, when discs in the spine of the human body abnormally function because of regression, aging, or accidents, an operation for removing an intervertebral disc (disc) of the diseased part and inserting a fusion cage to fuse vertebral bodies is performed.

This fusion cage may be provided in plurality between the vertebral bodies according to the damaged degree and range of the disc. Here, it is very important that the fusion cage has to not only perform its original function for fixing the vertebral bodies without being separated from the vertebral bodies, but also minimize the damages of upper and lower vertebral bodies and well support the vertebral bodies to prevent the upper and lower vertebral bodies from collapsing.

Referring to the related arts regarding to the fusion cage, e.g., Korean Patent Registration No. 10-1040515, which is entitled “FUSION CAGE FOR FIXING INTERVERTEBRAL BODIES” and Korean Patent Registration No. 10-1388078, which is entitled “CAGE HAVING BLADES FOR FIXING INTERVERTEBRAL BODIES”, a fusion cage inserted between intervertebral bodies is firmly fixed to the vertebral body by using a screw disposed on each of upper and lower portions of the fusion cage, or a blade exposed to upper and lower portions of a fusion cage is caught in vertebral bodies so that the fusion cage is fixed between the vertebral bodies.

According to the related arts, in the actual operation, it may be difficult to insert the screw into the human body due to an insertion angle thereof, and an additional process in which a separate blade is inserted may be cumbersome. Also, when a plurality of fusion cages are inserted into the vertebral bodies to form multiple layers, the screws or blades of the fusion cages adjacent to each other overlap close to each other, and thus the vertebral bodies may be cracked. In severe cases, the vertebral bodies may be broken.

Therefore, there is a desperate need for a safer fusion cage capable of well protecting the spine while faithfully performing its original function.

### PRIOR ART DOCUMENT

### [Patent Document]

Korean Patent Registration No. 10-1040515

Korean Patent Registration No. 10-1388078

### SUMMARY

Embodiments provide an intervertebral fusion cage combined with a fixing blade, which is capable of being inserted between vertebral bodies to prevent the fusion cage from moving, thereby increasing a bone fusion rate, and capable of easily being inserted and fixed without an additional operation process.

Embodiments also provide an intervertebral fusion cage combined with a fixing blade, which is capable of supporting upper and lower vertebral bodies to prevent a phenomenon in which the upper and lower vertebral bodies collapse from occurring.

Embodiments also provide an intervertebral fusion cage combined with a fixing blade, in which fixing blades protruding from upper and lower portions of the fusion cage are disposed on left and right sides of a fusion cage body to prevent a phenomenon, in which vertebral bodies are cracked or broken because the fixing blades overlap each other when a plurality of fusion cages are inserted, from occurring.

In one embodiment, an intervertebral fusion cage combined with a fixing blade, which is inserted to vertebral bodies adjacent to each other, instead of an intervertebral disc, the intervertebral fusion cage includes: a fusion cage body having a bone fusion hole passing from a top surface to a bottom surface thereof, the fusion cage body being inserted between the vertebral bodies adjacent to each other; an upper vertebral body coupling unit coupled to a first accommodation part defined inward from one side of a front portion of the fusion cage body, the upper vertebral body coupling unit having a lower portion that is coupled to the first accommodation part and an upper portion that is exposed to an upper side of the fusion cage body and provided with a first fixing blade inserted into an upper vertebral body; and a lower vertebral body coupling unit coupled to a second accommodation part defined inward from the other side of the front portion of the fusion cage body, the lower vertebral body coupling unit having an upper portion that is coupled to the second accommodation part and a lower portion that is exposed to a lower side of the fusion cage body and provided with a second fixing blade inserted into a lower vertebral body.

In another embodiment, the upper and lower vertebral coupling units may be disposed at positions that are spaced a predetermined distance from each other with respect to a central axis in a direction to which the fusion cage body is inserted.

In further embodiment, the upper vertebral body coupling unit may have a ‘ [symbol] ’ shape and be provided with the first fixing blade that is disposed on the upper portion thereof and inserted to one side of the upper vertebral body, and the lower vertebral body coupling unit may have a ‘ [symbol] ’ shape that is an upside-down shape of the upper vertebral body coupling unit and be provided with the second fixing blade that is disposed on the lower portion thereof and inserted to the other side of the lower vertebral body, which is disposed at a position symmetrical to the one side of the upper vertebral body with respect to the central axis in the direction to which the fusion cage body is inserted.

In further embodiment, the fusion cage body may have a through-hole passing through both side surfaces of the front portion thereof, and the upper vertebral body coupling unit may include: a first coupling part coupled to the first accommodation part and in which a through-hole is defined; a first fixing blade protruding in a direction in which the upper vertebral body coupling unit is inserted toward the upper vertebral body; and a connection part disposed on an upper portion of the first coupling part to connect the first coupling part to the first fixing blade, wherein the first coupling part is inserted to the first accommodation part, and the upper vertebral body coupling unit is coupled to the fusion cage body by a first coupling pin passing through the through-holes defined in the fusion cage body and the first coupling part, and the lower vertebral body coupling unit may include: a second coupling part coupled to the second accommodation part and in which a through-hole is defined; a second fixing blade protruding in a direction in which the lower vertebral body coupling unit is inserted toward the lower vertebral body; and a connection part disposed on a lower portion of the second coupling part to connect the second coupling part to the second fixing blade, wherein the second coupling part is inserted to the second accommodation part, and the lower vertebral body coupling unit is coupled to the fusion cage body by a second coupling pin passing through the through-holes defined in the fusion cage body and the second coupling part.

In further embodiment, each of the first and second fixing blades may have a plate shape to support the upper and lower vertebral bodies, thereby preventing the upper and lower vertebral bodies from collapsing, and a sharp blade may be disposed on a front portion, which is inserted first to the vertebral body, of each of the first and second fixing blades.

In further embodiment, a protrusion may be disposed on one surface of each of the plate-shaped first and second fixing blades to prevent each of the first and second fixing blades from slipping off after each of the first and second fixing blades is inserted to the vertebral body.

The details of one or more embodiments are set forth in the accompanying drawings and the description below. Other features will be apparent from the description and drawings, and from the claims.

### BRIEF DESCRIPTION OF THE DRAWINGS

FIG. 1A is a first perspective view of an intervertebral fusion cage combined with a fixing blade according to an embodiment.

FIG. 1B is a second perspective view of the intervertebral fusion cage combined with a fixing blade according to an embodiment.

FIG. 2 is a front view of the intervertebral fusion cage combined with the fixing blade according to an embodiment.

FIG. 3 is a side view of the intervertebral fusion cage combined with the fixing blade according to an embodiment.

FIG. 4 is a plan view of the intervertebral fusion cage combined with the fixing blade according to an embodiment.

FIG. 5 is an exploded perspective view of the intervertebral fusion cage combined with the fixing blade according to an embodiment.

FIG. 6 is an exemplary view illustrating a state where force for preventing upper and lower vertebral bodies from collapsing between vertebral bodies acts on the fusion cage combined with the fixing blade according to an embodiment.

FIG. 7A is a view illustrating a state where the intervertebral fusion cage combined with the fixing blade is being inserted between the vertebral bodies.

FIG. 7B is a view illustrating a state after the intervertebral fusion cage combined with the fixing blade is inserted between the vertebral bodies.

FIG. 7C is a view illustrating a state where the fixing blade is disposed at a position that is spaced inward from a fusion cage body by a predetermined length.

FIG. 7D is a view illustrating a state where the fixing blades of the fusion cages do not overlap each other when a plurality of intervertebral fusion cages combined with the fixing blades are inserted between the vertebral bodies.

### DETAILED DESCRIPTION OF THE EMBODIMENTS

Since the present disclosure may implement diverse modifications and have many embodiments, specific embodiments are illustrated in the drawings and are described in detail in the detailed description.

However, it is not the intention to limit the present disclosure to specific embodiments and it should be understood that the present disclosure covers all modifications, equivalents, and replacements within the idea and technical scope of the present disclosure.

In the description of the drawings, like reference numerals refer to like elements throughout. It will be understood that although the terms “first” and “second” are used herein to describe various elements, these elements should not be limited by these terms. The terms are only used to distinguish one component from other components.

For example, the word “and/or” means that one or more or a combination of relevant constituent elements is possible.

Unless terms used in the present disclosure are defined differently, the terms may be construed to have a meaning known to those skilled in the art.

Terms such as those that are generally used and are defined in dictionaries should be construed as having meanings matching contextual meanings in the art. In this description, unless defined clearly, terms are not to be interpreted as having ideally or excessively formal meanings.

Hereinafter, embodiments according to the present disclosure will be described in detail with reference to the accompanying drawings.

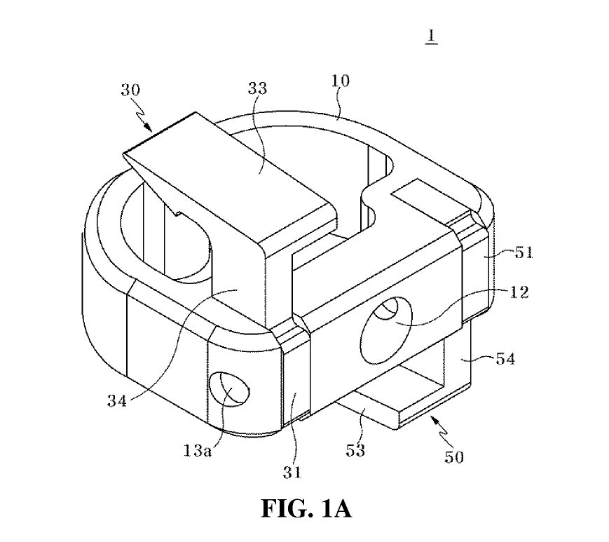

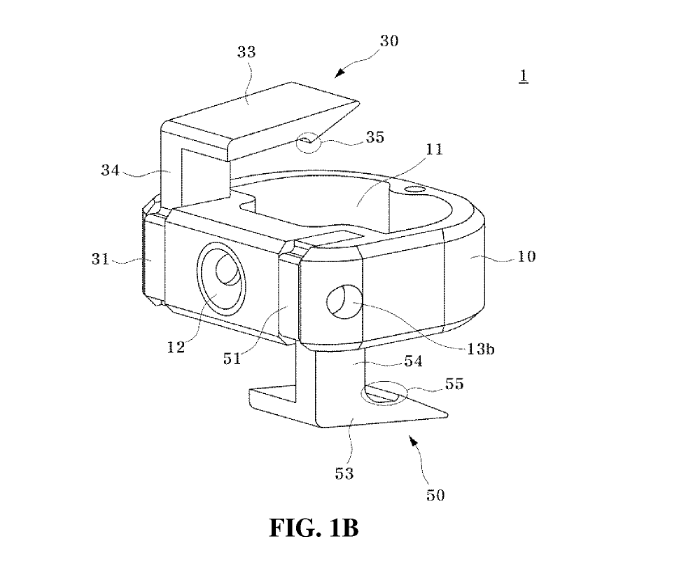

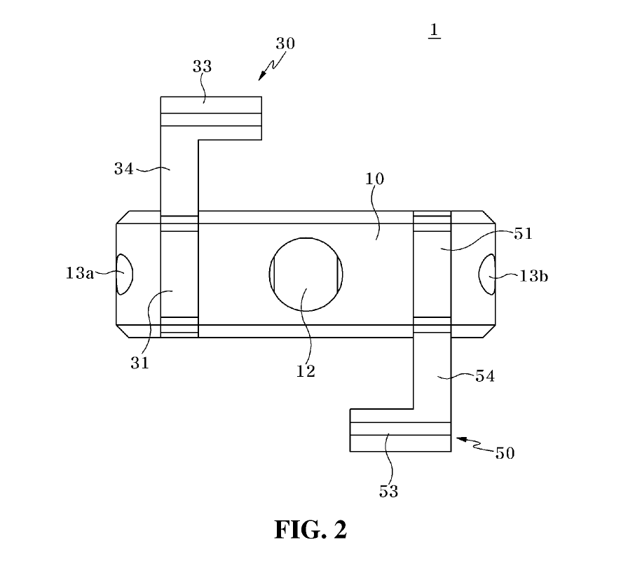

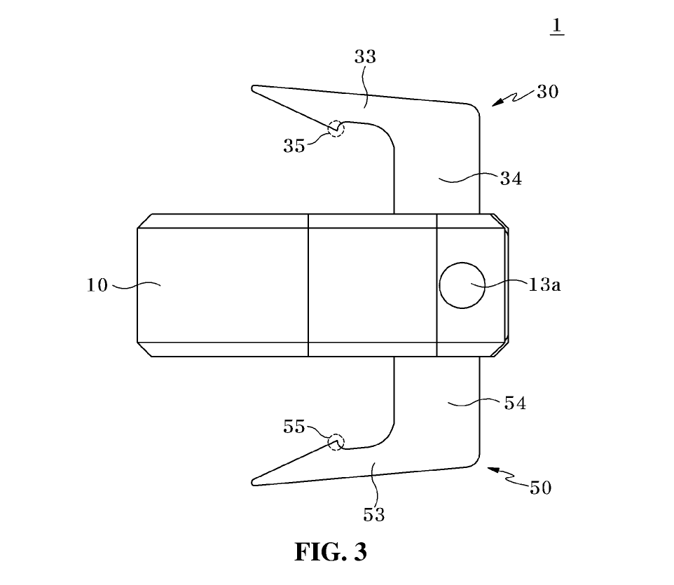

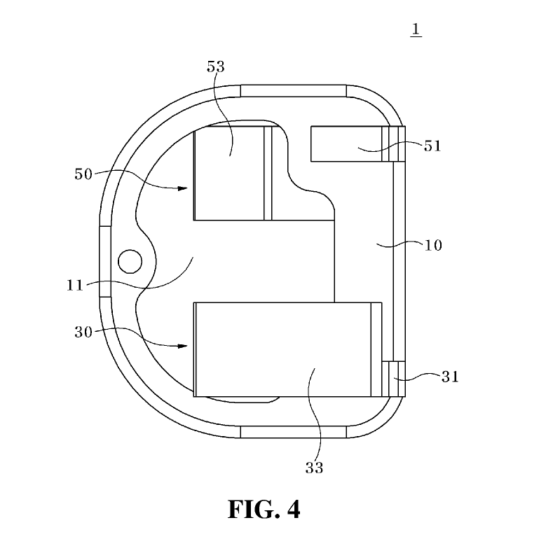

Referring to FIGS. 1A to 5, an intervertebral fusion cage 1 combined with a fixing blade (hereinafter, referred to as an “intervertebral fusion cage”) is a fusion cage that is inserted between vertebral bodies adjacent to each other, instead of an intervertebral disc (disc). The intervertebral fusion cage 1 includes a fusion cage body 10, an upper vertebral body coupling unit 30, and a lower vertebral body coupling unit 50.

The fusion cage body 10 has a bone fusion hole 11 passing from a top surface to a bottom surface thereof. After an operation, an autogenous bone or a bone substitute material is filled in the bone fusion hole 11 to fuse bones. A fusion cage insertion tool is coupled to a tool coupling hole 12 defined in a front portion of the fusion cage body 10, and then the fusion cage body 10 is inserted between the vertebral bodies.

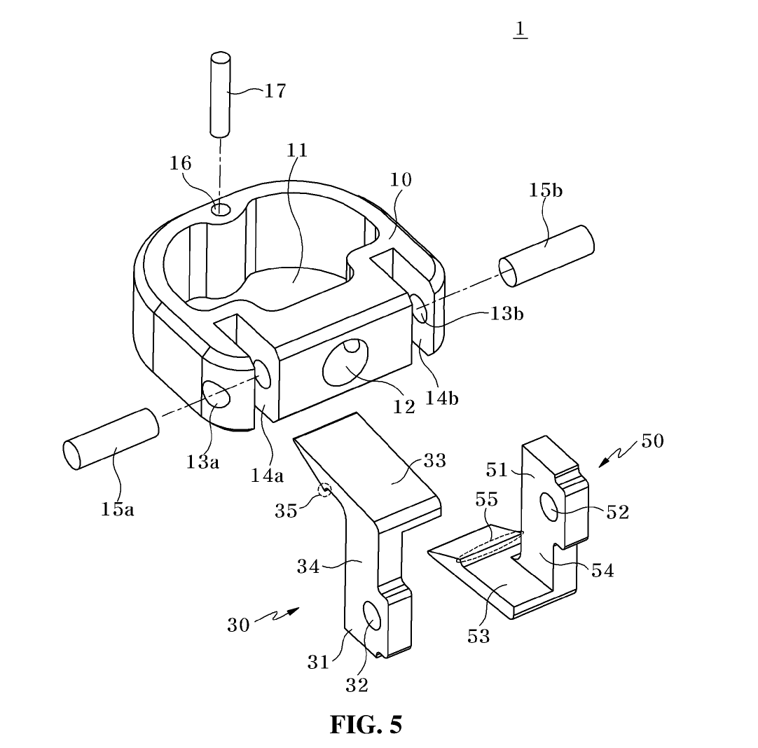

Referring to FIG. 5, a marker pin 17 is inserted to an insertion hole 16. A pin through which X-rays are not transmitted so that a position of the fusion cage 1 is checked through the X-rays during and after the operation may be used as the marker pin 17.

The upper vertebral coupling unit 30 is coupled to a first accommodation part 14a defined inward from one side of a front portion of the fusion cage body 10. Here, the upper vertebral coupling unit 30 has a lower portion that is coupled to the first accommodation part 14a and an upper portion that is exposed to an upper side of the fusion cage body 10. The upper portion of the upper vertebral coupling unit 30 is provided with a first fixing blade 33 inserted to an upper vertebral body.

The lower vertebral body coupling unit 50 is coupled to a second accommodation part 14b defined inward from the other side of the front portion of the fusion cage body 10. Here, the lower vertebral body coupling unit 50 has an upper portion that is coupled to the second accommodation part 14b and a lower portion that is exposed to a lower side of the fusion cage body 10. The lower portion of the upper vertebral coupling unit 30 is provided with a second fixing blade 53 inserted to a lower vertebral body.

In detail, the upper and lower vertebral body coupling units 30 and 50 are disposed to be spaced a predetermined distance from each other with respect to a central axis in a direction to which the fusion cage body 10 is inserted. When the central axis passes through a center of the tool coupling hole 12, the upper vertebral body coupling unit 30 may be disposed in the first accommodation part 14a that is defined to be spaced a predetermined distance from the central axis to a left side, and the lower vertebral body coupling unit 50 may be disposed in the second accommodation part 14b that is defined to be spaced a predetermined distance from the central axis to a right side.

The upper vertebral body coupling unit 30 has a “ [symbol] ” shape. The first fixing blade 33 disposed on the upper portion of the upper vertebral body coupling unit 30 may be inserted into one side of the upper vertebral body. The lower vertebral body coupling unit 50 has a “ [symbol] ” shape that is an upside-down shape of the upper vertebral body coupling unit 30. The second fixing blade 53 disposed on the lower portion of the lower vertebral body coupling unit 50 may be inserted into the other side of the lower vertebral body, which is disposed at a position symmetrical to the one side of the upper vertebral body with respect to the central axis in the direction to which the fusion cage body 10 is inserted.

For example, the first fixing blade 33 disposed on the upper portion of the “ [symbol] ”-shaped upper vertebral body coupling unit 30 may be inserted to a left side of the upper vertebral body. Also, the second fixing blade 53 disposed on the lower portion of the lower vertebral body coupling unit 50 having the “ [symbol] ” shape that is the upside-down shape of the upper vertebral body coupling unit 30 may be inserted to a right side of the lower vertebral body.

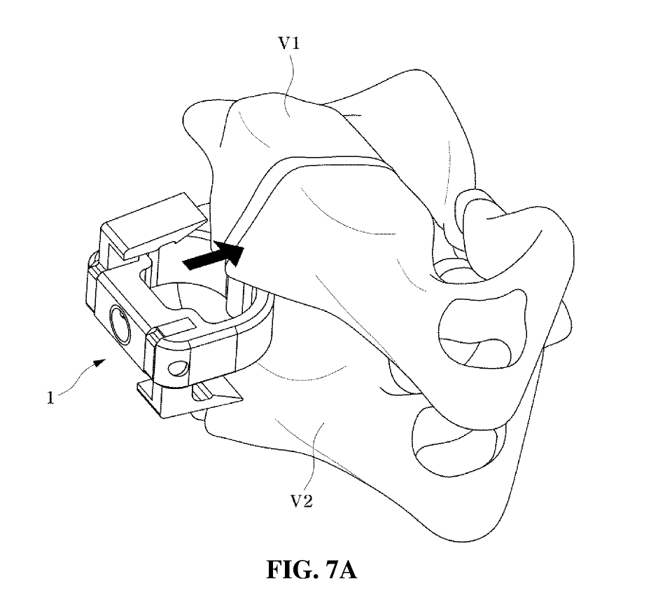

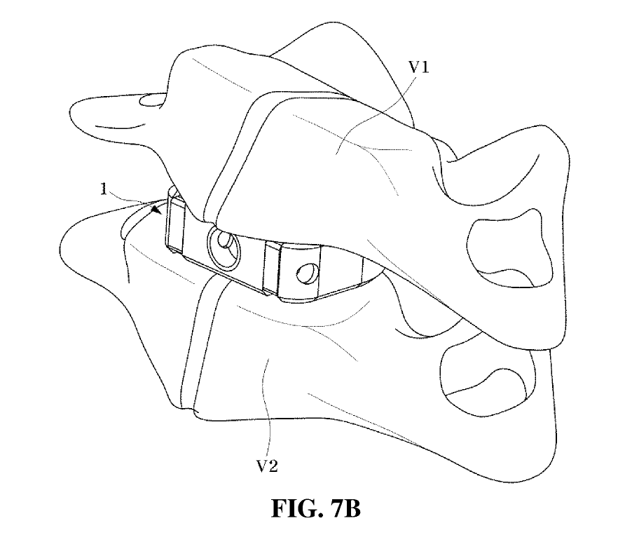

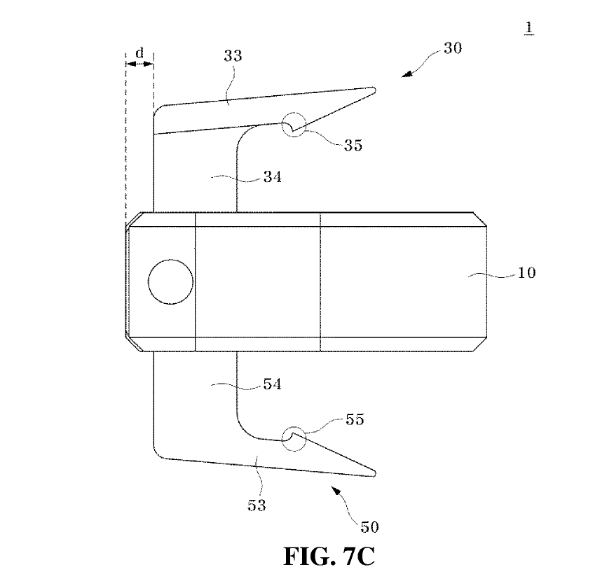

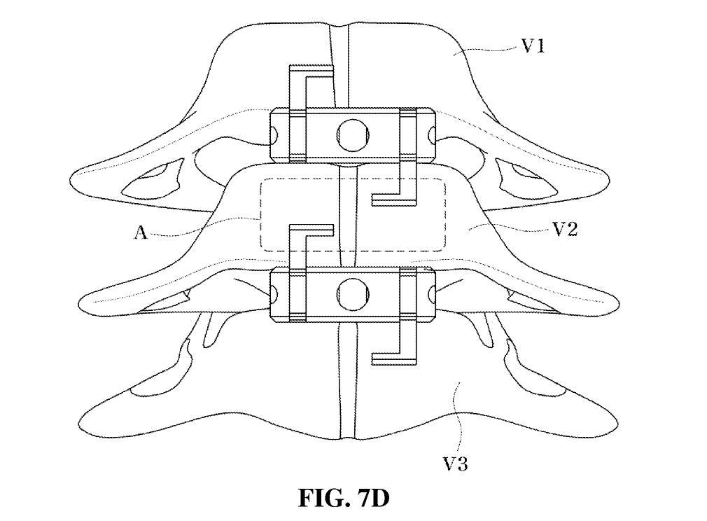

Referring to FIG. 7D, the intervertebral fusion cage 1 according to an embodiment is inserted between vertebral bodies V1 and V2, and another fusion cage 1 is inserted between vertebral bodies V2 and V3. Referring to an area A, the first fixing blade 33 of the upper vertebral body coupling unit 30 and the second fixing blade 53 of the lower vertebral body coupling unit 50 are inserted to left and right sides of the area A, which are positions symmetrical to each other with respect to the central axis in the direction to which the fusion cage body 10 is inserted. According to the structure, the phenomenon in which the vertebral bodies are cracked or broken due to the collision between the fixing blades of the fusion cages 1 different from each other or the concentration of the fixing blades to the central portions of the vertebral bodies may be prevented.

The intervertebral fusion cage 1 according to an embodiment will be described in more detail with reference to FIG. 5.

In the fusion cage body 10 has through-holes 13a and 13b passing through both side surfaces of the front portion of the fusion cage body 10. The left through-hole 13a passes from the front left surface of the fusion cage body 10 to the tool coupling hole 12. The right through-hole 13b passes from the front right surface of the fusion cage body 10 to the tool coupling hole 12.

The first and second accommodation parts 14a and 14b recessed inward from the fusion cage body 10 are defined in the front left and right sides of the fusion cage body 10. The lower portion of the upper vertebral body coupling unit 30 is inserted to the first accommodation part 14a, and the upper portion of the lower vertebral body coupling unit 50 is inserted to the second accommodation part 14b.

The upper vertebral body coupling unit 30 includes a first coupling part 31 coupled to the first accommodation part 14a and in which a through-hole 32 is defined, the first fixing blade 33 protruding in direction where the upper vertebral body coupling unit 30 is inserted toward the upper vertebral body, and a connection part 34 disposed on an upper portion of the first coupling part 31 to connect the first coupling part 31 to the first fixing blade 33. The first coupling part 31 is inserted to the first accommodation part 14a, and the upper vertebral body coupling unit 30 is coupled to the fusion cage body 10 by a first coupling pin 15a passing through the through-holes 13a and 32 defined in the fusion cage body 10 and the first coupling part 31.

The lower vertebral body coupling unit 50 includes a second coupling part 51 coupled to the second accommodation part 14b and in which a through-hole 52 is defined, the second fixing blade 53 protruding in a direction where the lower vertebral body coupling unit 50 is inserted toward the lower vertebral body, and a connection part 54 disposed on a lower portion of the second coupling part 51 to connect the second coupling part 51 to the second fixing blade 53. The second coupling part 51 is inserted to the second accommodation part 14b, and the lower vertebral body coupling unit 50 is coupled to the fusion cage body 10 by a second coupling pin 15b passing through the through-holes 13b and 52 defined in the fusion cage body 10 and the second coupling part 51.

The first and second coupling parts 31 and 51 have shapes corresponding to those of the accommodation parts 14a and 14b, respectively. The connection part 34 is vertically disposed on the upper portion of the coupling part 31, and the connection part 54 is vertically disposed on the lower portion of the coupling part 51. Here the fixing blades 33 and 53 are disposed on ends of the connection parts 34 and 54, respectively.

Also, each of the first and second fixing blades 33 and 53 may have a rectangular plate shape so that the first and second fixing blades 33 and 53 support the upper and lower vertebral bodies to prevent the upper and lower vertebral bodies from collapsing, respectively. Here, a sharp blade is disposed on a front portion, which is inserted first to the vertebral body, of each of the first and second fixing blades 33 and 53. Also, each of protrusions 35 and 55 is disposed on one surface of the plate shape of each of the first and second fixing blades 33 and 53 to prevent each of the first and second fixing blades 33 and 53 from slipping off after each of the first and second fixing blades 33 and 53 is inserted to the vertebral body.

In detail, each of the protrusions 35 and 55 may be disposed on an end of the sharp blade of the front portion, which is inserted first to the vertebral body, of each of the fixing blades 33 and 53 so that each of the fixing blades 33 and 53 does not slip off after each of the fixing blades 33 and 53 is inserted to the vertebral body.

Referring to FIG. 7C, the first fixing blade 33 disposed on the upper vertebral body coupling unit 30 is inclined so that the front portion of the first fixing blade 33 is higher than a rear portion of the first fixing blade 33 to prevent the first fixing blade 33 from slipping off after the first fixing blade 33 is inserted to the bone. Likewise, the second fixing blade 53 disposed on the lower vertebral body coupling unit 50 is inclined so that the front portion of the second fixing blade 53 is lower than a rear portion of the second fixing blade 53.

Referring to FIGS. 7B and 7C, when the intervertebral fusion cage 1 is inserted between the vertebral bodies V1 and V2, the fixing blades 33 and 53 and the connection parts 34 and 54 may be buried in the bones to pass out of sight while fusing with the bones as time elapses. When the fusion cage 1 is viewed from a side, each of the connection parts 34 and 54 is disposed inward by a predetermined distance d from the foremost portion of the fusion cage body 10. Each of the connection parts 34 and 54 and each of the fixing blades 33 and 53 are disposed inward by the predetermined distance from the foremost portion of the fusion cage body 10. Thus, after the fusion cage 1 is inserted to the vertebral bodies V1 and V2, a portion of the fusion cage 1 except for the fusion cage body 10 may fuse with the bones to pass out of sight. According to the structure, the fusion cage 1 inserted between the vertebral bodies V1 and V2 may be further firmly fixed without being separated.

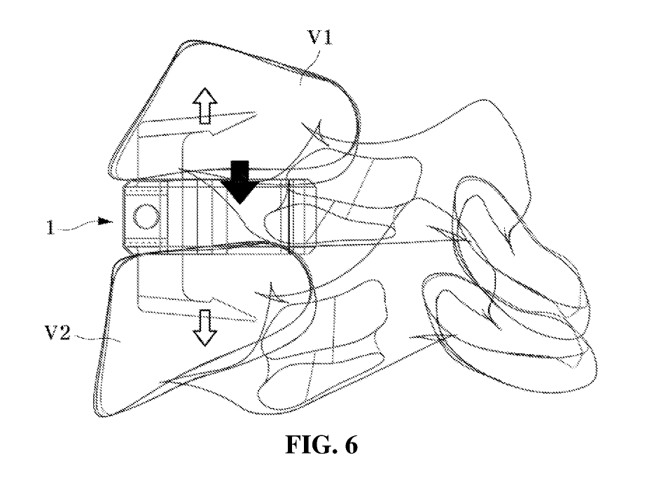

Referring to FIG. 6, the rectangular plate-shaped first fixing blade 33 inserted between the vertebral bodies V1 and V2 may push and support the upper vertebral body V1 upward to prevent the upper vertebral body V1 from collapsing, and the second fixing blade 53 may hold the lower vertebral body V2 to prevent the lower vertebral body V2 from collapsing.

According to the intervertebral fusion cage combined with the fixing blade, the intervertebral fusion cage may be inserted between the vertebral bodies to prevent the fusion cage from moving by using the fixing blades thereon, thereby increasing the bone fusion rate and may be easily inserted and fixed without the additional operation process.

Also, the intervertebral fusion cage combined with the fixing blade may support the upper and lower vertebral bodies to prevent the upper and lower vertebral bodies from collapsing.

Also, the fixing blades protruding from upper and lower portions of the fusion cage may be disposed on the left and right sides, which are spaced a predetermined distance from the center of the fusion cage, to prevent the phenomenon in which the vertebral body is cracked or broken due to the overlapping of the fixing blades overlap when the plurality of fusion cages are inserted from occurring.

Although embodiments have been described with reference to a number of illustrative embodiments thereof, it should be understood that numerous other modifications and embodiments can be devised by those skilled in the art that will fall within the spirit and scope of the principles of this disclosure.

What is claimed is:

1. An intervertebral fusion cage combined with a fixing blade, which is inserted to vertebral bodies adjacent to each other, instead of an intervertebral disc, the intervertebral fusion cage comprising:
a fusion cage body having a bone fusion hole passing from a top surface to a bottom surface thereof, the fusion cage body being inserted between the vertebral bodies adjacent to each other;
an upper vertebral body coupling unit coupled to a first accommodation part defined inward from one side of a front portion of the fusion cage body, the upper vertebral body coupling unit having a lower portion that is coupled to the first accommodation part and an upper portion that is exposed to an upper side of the fusion cage body and provided with a first fixing blade inserted into an upper vertebral body; and
a lower vertebral body coupling unit coupled to a second accommodation part defined inward from the other side of the front portion of the fusion cage body, the lower vertebral body coupling unit having an upper portion that is coupled to the second accommodation part and a lower portion that is exposed to a lower side of the fusion cage body and provided with a second fixing blade inserted into a lower vertebral body.

2. The intervertebral fusion cage of claim 1, wherein the upper and lower vertebral coupling units are disposed at positions that are spaced a predetermined distance from each other with respect to a central axis in a direction to which the fusion cage body is inserted.

3. The intervertebral fusion cage of claim 1, wherein the upper vertebral body coupling unit has a ‘ [symbol] ’ shape and is provided with the first fixing blade that is disposed on the upper portion thereof and inserted to one side of the upper vertebral body, and
the lower vertebral body coupling unit has a ‘ [symbol] ’ shape that is an upside-down shape of the upper vertebral body coupling unit and is provided with the second fixing blade that is disposed on the lower portion thereof and inserted to the other side of the lower vertebral body, which is disposed at a position symmetrical to the one side of the upper vertebral body with respect to the central axis in the direction to which the fusion cage body is inserted.

4. The intervertebral fusion cage of claim 1, wherein the fusion cage body has a through-hole passing through both side surfaces of the front portion thereof, and
the upper vertebral body coupling unit comprises:
a first coupling part coupled to the first accommodation part and in which a through-hole is defined;
a first fixing blade protruding in a direction in which the upper vertebral body coupling unit is inserted toward the upper vertebral body; and
a connection part disposed on an upper portion of the first coupling part to connect the first coupling part to the first fixing blade, wherein the first coupling part is inserted to the first accommodation part, and the upper vertebral body coupling unit is coupled to the fusion cage body by a first coupling pin passing through the through-holes defined in the fusion cage body and the first coupling part, and
the lower vertebral body coupling unit comprises:
a second coupling part coupled to the second accommodation part and in which a through-hole is defined;
a second fixing blade protruding in a direction in which the lower vertebral body coupling unit is inserted toward the lower vertebral body; and
a connection part disposed on a lower portion of the second coupling part to connect the second coupling part to the second fixing blade, wherein the second coupling part is inserted to the second accommodation part, and the lower vertebral body coupling unit is coupled to the fusion cage body by a second coupling pin passing through the through-holes defined in the fusion cage body and the second coupling part.

5. The intervertebral fusion cage of claim 1, wherein each of the first and second fixing blades has a plate shape to support the upper and lower vertebral bodies, thereby preventing the upper and lower vertebral bodies from collapsing, and
a sharp blade is disposed on a front portion, which is inserted first to the vertebral body, of each of the first and second fixing blades.

6. The intervertebral fusion cage of claim 5, wherein a protrusion is disposed on one surface of each of the plate-shaped first and second fixing blades to prevent each of the first and second fixing blades from slipping off after each of the first and second fixing blades is inserted to the vertebral body.
